(19) United States
(12) Patent Application Publication
(10) Pub. No.: US 2016/0089151 A1
(43) Pub. Date: Mar. 31, 2016

(54) LEFT ATRIAL APPENDAGE OCCLUSION DEVICES AND METHODS

(71) Applicant: CEDARS-SINAI MEDICAL CENTER, Los Angeles, CA (US)

(72) Inventors: Robert James Siegel, Beverly Hills, CA (US); Matthew J. Price, La Jolla, CA (US); Nina Wunderlich, Frankfurt (DE)

(73) Assignee: Cedars-Sinai Medical Center, Los Angeles, CA (US)

(21) Appl. No.: 14/893,418

(22) PCT Filed: May 23, 2014
(86) PCT No.: PCT/US2014/039438

§ 371 Date: Nov. 23, 2015

Related U.S. Application Data
(60) Provisional application No. 61/827,354, filed on May 24, 2013.

(51) Int. Cl. A61B 17/12

(52) U.S. Cl. CPC A61B 17/12186; A61B 17/12122; A61B 17/12136; A61B 17/12195; A61B 2017/12054; A61B 2017/22042

(57) ABSTRACT

A catheter device is provided that includes an elongate body, an atraumatic member, an expandable member, and a locking device. The elongate body has a fluid flow lumen that is in fluid communication with an outlet port adjacent to a distal end of the elongate body. The atraumatic member can be at the tip of the elongate body. The expandable member is disposed proximal of the atraumatic tip and is configured to block an opening of the LAA. The locking device is disposed adjacent to the expandable member. The locking device has a first configuration in which the elongate body is coupled with the atraumatic member and second configuration in which the elongate body is uncoupled from the atraumatic member.

### BACKGROUND OF THE INVENTION

1. Field of the Invention

This application is directed to methods and devices that can be used to occlude a left atrial appendage.

2. Description of the Related Art

The left atrium has a blind-ended structure connected to it called the left atrial appendage (LAA). Blood flows between the left atrium and the LAA in the normal operation of the heart.

A stroke is a potentially deadly event that arise when a blood clot in the blood stream blocks critical blood vessels. Even if not fatal, strokes can seriously degrade critical organs, greatly affecting the stroke patient's life. Studies have estimated that more than 15% of strokes originate in the heart and from the LAA in particular.

Atrial fibrillation is a common cardiac arrhythmia (irregular heart beat). In atrial fibrillation, the lack of an organized atrial contraction can result in some stagnant blood in the left atrium or the LAA. This lack of movement of blood can lead to thrombus formation, or blood clots. Emboli (mobile thrombus or clots) in the brain may result in an ischemic stroke or a transient ischemic attack (TIA). More than 90% of cases of thrombi associated with non-valvular atrial fibrillation evolve in the left atrial appendage.

Although the concept of LAA occlusion has been discussed as a way to reduce the risk of stoke based on LAA originating thrombus, with various devices proposed to achieve such occlusion, such devices have not gained wide use.

### SUMMARY OF THE INVENTION

Existing devices are unable to provide successful LAA occlusion therapy for a number of reasons. The devices that have been developed are too large, too complex and/or do not have the requisite ease of use to provide a meaningful LAA occlusion option. The new inventions disclosed herein markedly advance this therapy.

In a first embodiment, a catheter device is provided. The catheter device includes an elongate body, an atraumatic member, an expandable member, and a locking device. The elongate body has a fluid flow lumen that extends therethrough. The fluid flow lumen is in fluid communication with an outlet port adjacent to a distal end of the elongate body. The atraumatic member is disposed at the distal end of the elongate body. The atraumatic member can be at the tip of the elongate body. In some cases, the atraumatic member can be in the form of a pigtail member. The expandable member is disposed proximal of the atraumatic tip and is configured to block an opening of the LAA. The locking device is disposed adjacent to the expandable member. The locking device has a first configuration in which the elongate body is coupled with the atraumatic member and second configuration in which the elongate body is un-coupled from the atraumatic member.

The locking device is located distal of the balloon in various embodiments, e.g., between the balloon and the atraumatic member. The locking device is located proximal of the balloon in various embodiments.

In another embodiment, a system for reducing the volume of a left atrial appendage is provided. The system includes a guidewire, a catheter device, and an in-situ curable polymer. The catheter device includes an elongate body, a pigtail member, a balloon, and a connection hub. The elongate body has a proximal portion, a distal portion, and a lumen extending through the proximal and distal portions. The lumen is in fluid communication with an outlet port adjacent to a distal end and an inlet port adjacent to a proximal end. The elongate body is configured to be advanced along the guidewire. The pigtail member disposed at the distal end of the elongate body and configured to be positioned within a left atrial appendage (LAA). The balloon disposed near the distal end of the elongate body. The balloon is configured to block an opening of the LAA when inflated. The connection hub is disposed along the elongate body and has a first configuration in which the proximal portion of the elongate body is coupled with the pigtail member. The connection hub has a second configuration in which at least the pigtail portion is un-coupled from the proximal portion of the elongate body. The in-situ curable polymer is adapted to be delivered through the lumen of the elongate body, out of the outlet port(s) distal the balloon.

In another embodiment, a method is provided for occluding a left atrial appendage (LAA) of a heart. In the method an access catheter is advanced through the venous vasculature into the right atrium. The septum between the left and right atria is crossed, e.g., by being punctured by a wire or catheter body. A guidewire is advanced through the septum into the LAA. An atraumatic member of a procedure catheter is advanced along the guidewire and into the LAA such that a plurality of outlets formed in the atraumatic member are distal the ostium of the LAA. The atraumatic member may be a tip member, e.g., in the form of a pigtail member. The configuration of the LAA can be evaluated. An expandable member is deployed from the procedure catheter to seal the LAA ostium. The sealed state of the LAA ostium is optionally confirmed. A sealant is caused to flow through the procedure catheter and out of the plurality of outlets formed in the atraumatic member into the LAA. After the sealant is secured in the LAA, the atraumatic member is detached from a proximal portion of the procedure catheter.

In another embodiment, a method for occluding a left atrial appendage (LAA) of a heart is provided. A guide member is positioned through the heart and into the LAA. An occlusion catheter system having an atraumatic member (e.g., a tip or pigtail) disposed at a distal portion thereof is advanced along the guide member such that the distal portion including the atraumatic member is disposed within the LAA. A fluid sealant is injected through the occlusion catheter system into the LAA. The fluid sealant is permitted to solidify in the LAA around the pigtail catheter to minimize flow of blood into the LAA. A proximal portion of the occlusion catheter system is detached from the atraumatic member.

### BRIEF DESCRIPTION OF THE DRAWINGS

A more complete appreciation of the subject matter of this application and the various advantages thereof can be realized by reference to the following detailed description, in which reference is made to the accompanying drawings in which:

FIG. 1 shows basic anatomy being treated in some of the methods disclosed herein;

FIG. 2 is a schematic plan and partial section view of a system that includes a catheter device;

FIG. 2A inset in FIG. 2 is a detail view of one embodiment of a connection hub;

FIG. 2B illustrates another connection hub in which minimal rotation is used to detach a distal portion from a proximal portion;

FIGS. 3 and 3A are schematics similar to those of FIGS. 2 and 2A illustrating another embodiment in which a system is provided that includes a detachable segment including a cap member;

FIG. 4 is a perspective view of a distal portion of a catheter device suitable for sealing a left atrial appendage (LAA);

FIG. 5 illustrates a first embodiment of a cap member that can be incorporated into a system herein;

FIG. 5A shows another embodiment of an expandable member (e.g., balloon) that can be deployed across an ostium in various embodiments;

FIG. 6 illustrates a second embodiment of a cap member that can be incorporated into a system herein;

FIG. 7 shows an early stage of a method in which a guide member is advanced though the intra-atrial septum and into the LAA:

FIG. 8 is a stage subsequent to that of FIG. 7 in which any of the catheter devices of FIGS. 2-6 is advanced along a guide member into the LAA;

FIG. 9 is a stage subsequent to that of FIG. 8 in which an expandable member is expanded into apposition with the anatomy around the ostium of the LAA;

FIG. 10 is a stage subsequent to that of FIG. 9 in which a curable agent, in the form of a fluid sealant has been injected into the LAA;

FIG. 11 is a stage subsequent to that of FIG. 10 in which the fluid sealant has been secured in the LAA, e.g., has been cured;

FIG. 12 is a stage subsequent to that of FIG. 11 in which a tip portion has been detached from a proximal portion of the catheter device of FIGS. 2-2A;

FIG. 12A shows the configuration of solidified sealant in the LAA in connection with a modified placement of the expandable member;

FIG. 13 is a stage subsequent to that of FIG. 12 in which the proximal portion of the catheter device of FIGS. 2-2A has been removed, leaving the atraumatic tip in place; and

FIG. 14 is a variation of the stage illustrated in FIG. 13, in which a balloon or other containment member is left behind after a catheter device of a delivery system is removed.

More detailed descriptions of various embodiments of LAA systems, components and methods useful to treat patients are set forth below.

### DETAILED DESCRIPTION OF THE PREFERRED EMBODIMENT

As noted above, treatment of the left atrial appendage would be a significant advance in prevention of certain types of stroke.

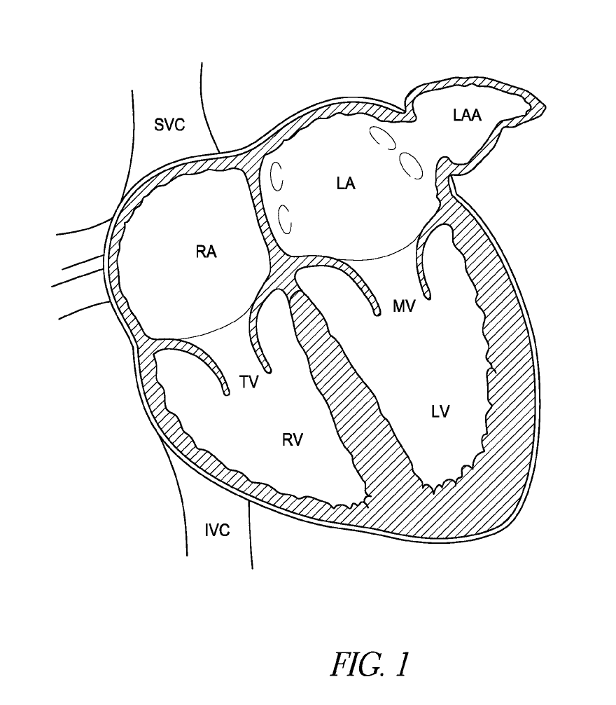

FIG. 1 shows relevant anatomy of the heart, reference below. The normal human heart has four chambers, i.e., the right and left atria (RA, LA) and the right and left ventricles (RV, LV). The RA receives blood from the venous vasculature. Venous blood enters the RA from the superior vena cava (SVC) and the inferior vena cava (IVC) blood vessels. Normal pumping of the heart causes blood in the RA to flow through the tricuspid valve (TV) into the right ventricle (RV). Blood in the RV is expelled from the heart into the pulmonary artery. Blood expelled into the pulmonary artery flows into the lungs where it is oxygenated and thereafter flows back to the LA. The oxygenated blood in the LA flows through the mitral valve (MV) into the LV. Blood in the LV is then expelled out of the heart into the ascending aorta and from there to smaller vessels of the systemic circulation. The RA and LA are separated by a wall of the heart, referred to as the atrial septum. FIG. 1 shows that the LAA extends off of the LA and is a blind-ended structure, as discussed above. The LAA can be eliminated or minimized by devices and methods discussed herein to reduce stroke risk among other benefits.

I. Systems for Reducing Flow into the LAA

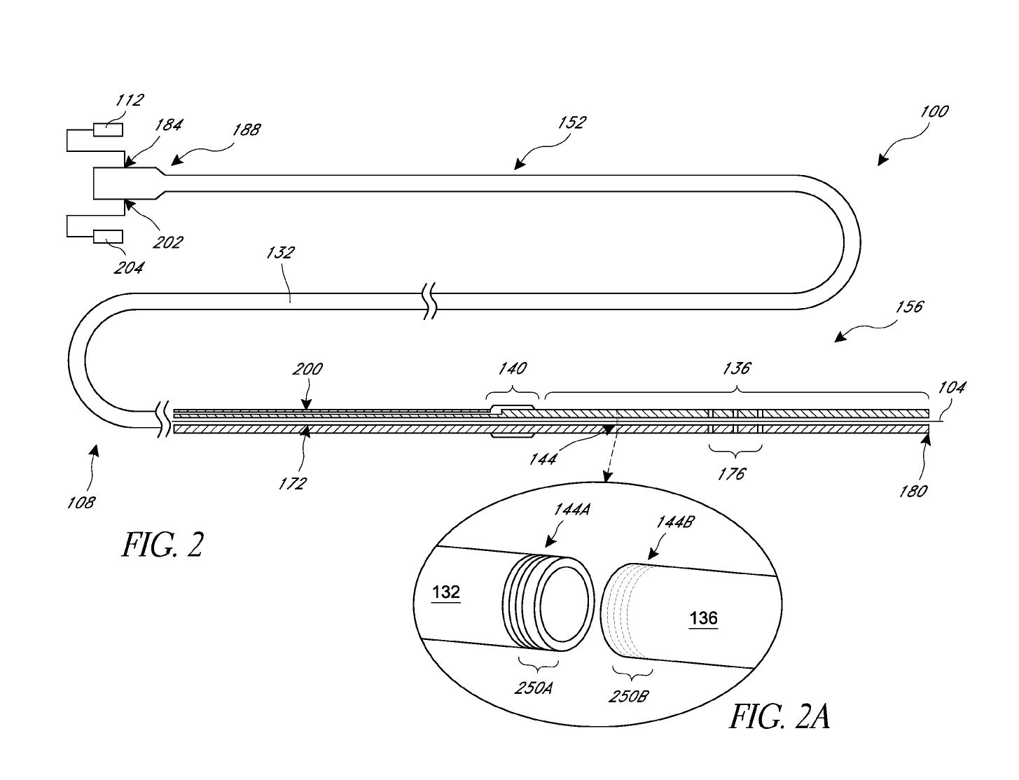

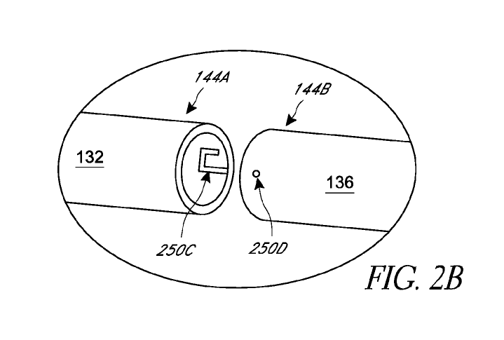

FIGS. 2 and 2A show various features of one embodiment of a system 100 for reducing flow into and/or the volume of the LAA. The system 100 includes a guidewire 104, a catheter device 108, and an in-situ curable agent 112, which can be a polymer alone or in combination with other structures or substances as discussed below. The guidewire 104 can be a standard guidewire device that is configured for accessing the LA from a venous approach, as discussed further below. The system 100 can also be configured without the guidewire 104, in which case it may be combined with a guide wire by the user. The curable agent 112 can be any suitable material that can be provided in a fluid state and cured within the body. More details of the agent 112 and its delivery are discussed below and in connection with various alternative for the same as described in U.S. Pat. No. 6,761,733, which is incorporated by reference and is included with this application in an Appendix.

The catheter device 108 includes an elongate body 132, an atraumatic member, which can be a pigtail member 136, a balloon 140, and a connection hub 144. The elongate body 132 has a proximal portion 152 and a distal portion 156. The proximal portion 152 preferably has one or more ports configured to be coupled with sources of fluid media. One fluid source can include an inflation medium for inflating the balloon 140. Another fluid source can include the curable agent 112. One or more ports can be provided for passing one or more guidewires.

In the illustrated embodiment a lumen 172 extends through the proximal and distal portions 152, 156 of the elongate body 132. The lumen 172 is in fluid communication with a plurality of outlet ports 176 adjacent to a distal end 180 and an inlet port 184 adjacent to a proximal end 188. Although several ports 176 are provided in the embodiment of FIG. 2, the devices 108 can be configured with as few as a single outlet port 176.

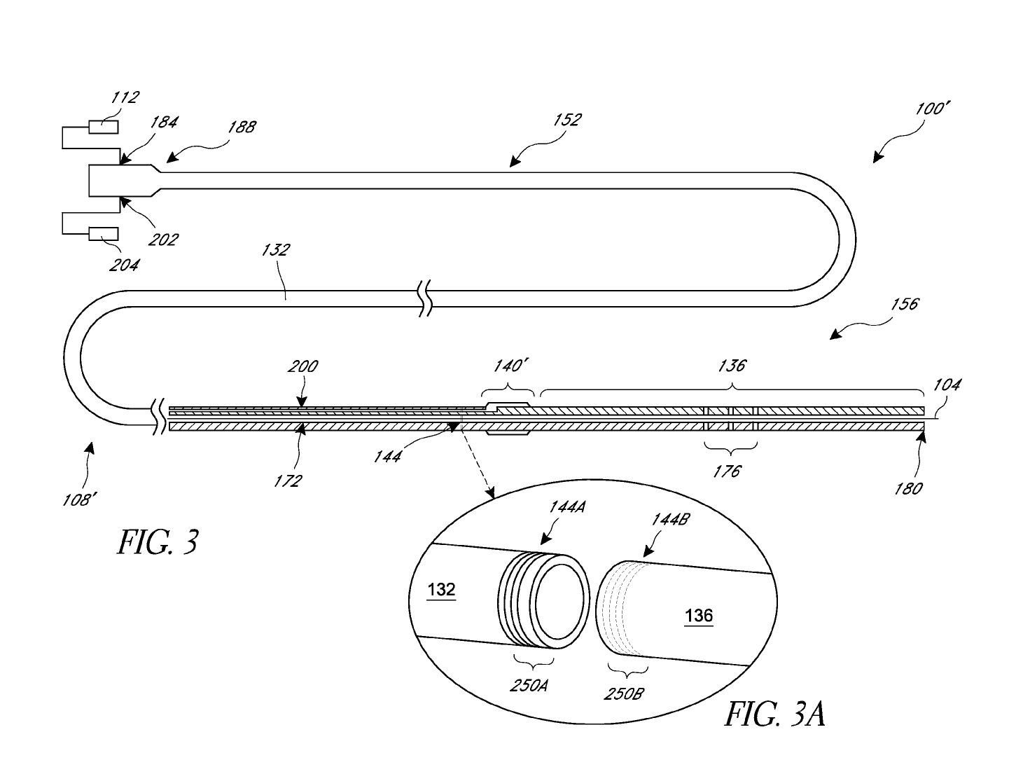

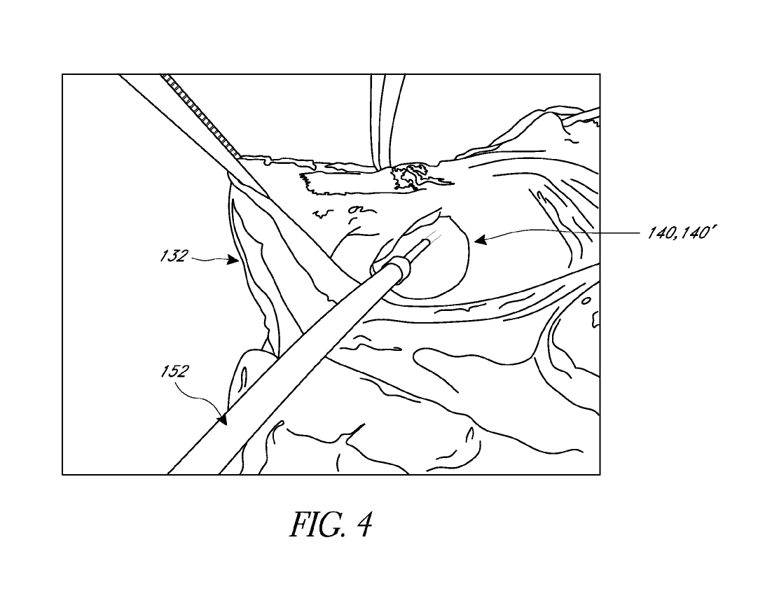

In the embodiments illustrated in FIGS. 2-3A, the lumen 172 also is used to move the guidewire 104 relative to the elongate body 132. As discussed further below, the elongate body 132 is configured to be advanced along the guidewire 104 after the guidewire is placed with a distal portion in the heart and a proximal portion at a venous access site.

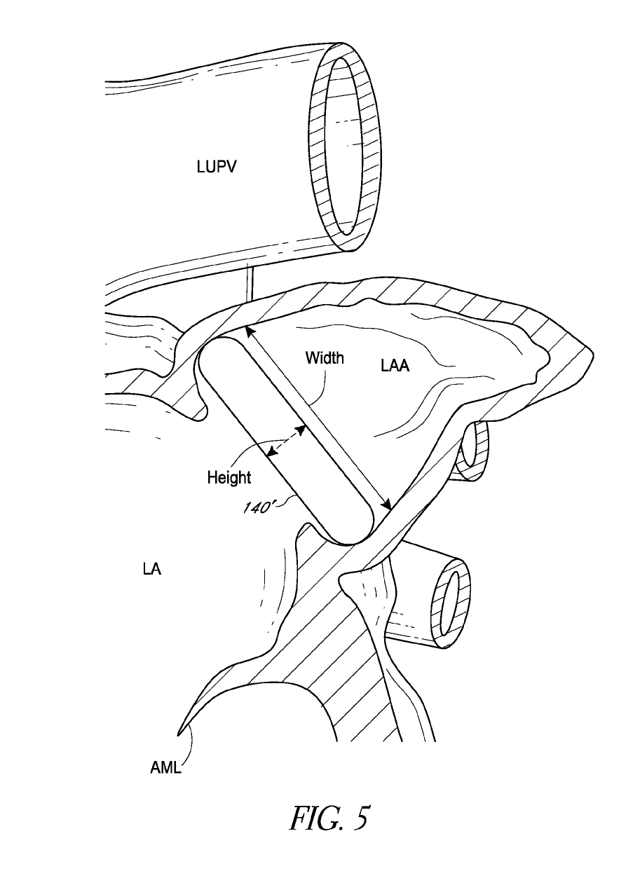

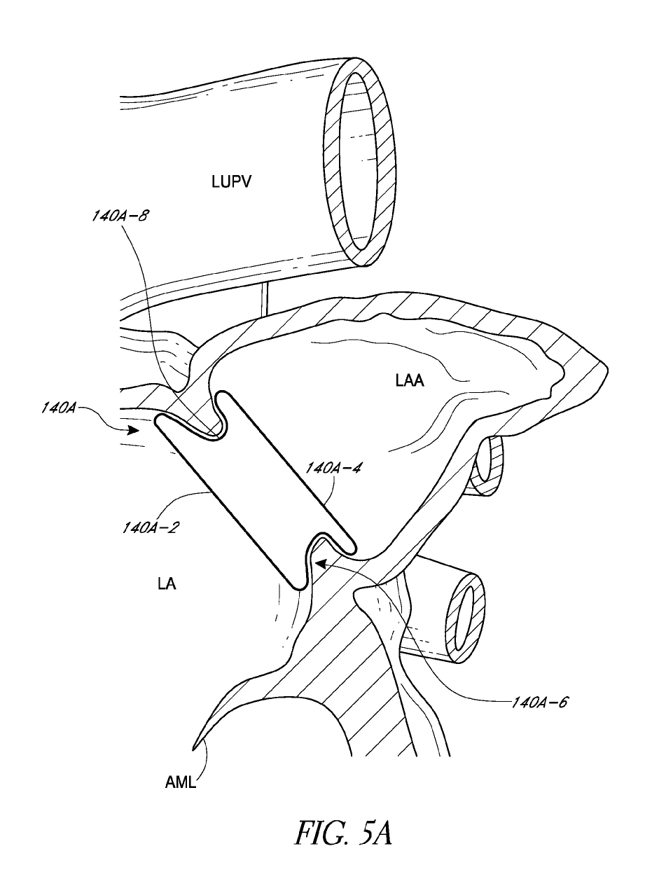

The pigtail member 136 is disposed at the distal end 180 of the elongate body 132. The pigtail member 136 can be positioned within the LAA, as discussed below. The pigtail member 136 is shown straight in FIG. 2. This configuration can arise from the higher stiffness of the guidewire 104, which straightens the pigtail 136. When deployed, the pigtail may have a tight curve as illustrated in FIG. 5. The size of the pigtail member 136 can vary, but in some embodiments about at 1 cm dimension is provided from inner edge to inner edge of the pigtail member. In other embodiments, the pigtail member 136 is a flexible member that can be disposed within a body structure having a transverse width of about 1 cm or less. Other devices that have been developed for occluding the LAA have been configured to include an anchor that attaches mechanically to a wall of the LAA. Such anchor is advanced into and in some cases through the wall of the LAA. While this approach enhances the anchoring of such devices, such anchor structures can pierce or breach the wall in some circumstances. A breach of the wall of the LAA can cause complications, such as cardiac tamponade, pericardial efflusion, bleeding, seepage of infused fluids, and/or uncontrolled tearing of the LAA wall. Each of these results can range in clinical effect from highly inconvenient to life threatening.

In contrast, the catheter devices disclosed herein are configured to preserve the containment capacity of the tissue surrounding the LAA. In particular, the pigtail member 136 has a soft, gentle curve that provides a soft interface with tissues within the LAA. Thus, the catheter device 108 not only does not require anchoring of an implant through the LAA wall, but is actually configured to maintain minimal contact with the LAA wall.

The balloon 140 is disposed near the distal end 180 of the elongate body 136. A lumen 200 disposed in the wall of the elongate body 136 provides fluid communication between a port 202 adjacent to the proximal end of the elongate body 136 and the interior of the balloon 140. The port 202 can be placed in fluid communication with a source of inflation media 204. The balloon 140 is configured to block an opening or ostium of the LAA when inflated.

FIGS. 2-6 illustrate several different embodiments of the system 100 and the balloon 140 discussed below. As discussed above, FIGS. 2-2A illustrate systems in which the balloon 140 is removed at the end of the procedure. A variety of balloon shapes are suitable for this approach. Although less critical for the embodiment of FIGS. 2-2A, the balloon 140 preferably is a low profile structure. In particular, a first side 212 of the balloon 140 is configured to be placed against the inside wall of the LA surrounding the ostium to the LAA or within the LAA. A second side 216 of the balloon faces the LA when the catheter device 108 is applied to the heart. A thickness of the balloon 140, as defined as the average distance between the first and second sides 212, 216 is preferably relatively small to not obstruct flow in the LA.

FIGS. 3-3A and 5-6 illustrate systems in which the balloon 140′ can be detached from a proximal portion of the catheter device 108′ and is left in place in the heart. FIG. 5 shows one embodiment of a balloon that is tire shaped, e.g., having a large ratio of diameter to height. In this context, “height” is the dimension transverse to the diameter, e.g., the distance along the axis of the LAA when the balloon 140′ is deployed. The balloon 140′ is preferably oversized compared to the size of the LAA. For example, the balloon 140′ can have an enlarged state that is configured to block the LAA. The enlarged state can be an inflated state that corresponds to an unconstrained inflated diameter that is about 10% greater than the width of the anatomy to which the balloon 140′ is to be apposed. With reference to FIG. 5, the LAA has a width and the balloon 140′ is inflated to a size that equals the width. Because the unconstrained diameter is larger than the width of the inner wall of the LAA, loads are applied by the balloon 140′ and the LAA on each other. In certain embodiments, the balloon 140′ has a generally circular outer perimeter with a radius in a range from about 3 mm to about 12 mm.

Other dimensions of the balloon 140′ that can be advantageously defined include the height, which as discussed above can be measured in a direction transverse to the width (e.g., diameter) of the balloon 140′. The height can be defined as the distance between the first and second sides 212, 216. The height is preferably kept relatively small to maximize the amount of the LAA that is filled. In some embodiments, the height of the balloon can be about 10 mm or less. In some embodiments, the balloon 140′ is adapted to permit tissue ingrowth so that the balloon will eventually not be exposed in the LA. For instance Dacron or other material suitable to encourage ingrowth of endothelial cells can be provided on the surface 216 for this purpose.

FIG. 5 shows that the balloon 140′ can be configured to be received entirely within the LAA in some embodiments, leaving a very short length of the LAA exposed to blood in the LA.

FIG. 5A shows a balloon 140A that can be deployed across the ostium, e.g., such that a proximal portion 140A-2 is disposed proximal of the ostium in the LA and a distal portion 140A-4 is disposed distal of the ostium inside the LAA. The proximal and distal portions 140A-2, 140A-4 can be disposed on opposite sides of a narrow portion 140A-6. In one embodiment, the balloon 140A is preformed such that when expanded to an expanded size, the narrow portion 140A-6 is configured to receive an anatomic narrows of the ostium between the LA and the LAA. In one embodiment, the narrow portion 140A-6 has a diameter or width that is no more than about 90% of the diameter or width of the smallest of the proximal portion 140A-2 and distal portions 140A-4. In one embodiment, the narrow portion 140A-6 has a diameter or width that is no more than about 80% of the diameter or width of the smallest of the proximal portion 140A-2 and distal portions 140A-4. In one embodiment, the narrow portion 140A-6 has a diameter or width that is no more than about 70% of the diameter or width of the smallest of the proximal portion 140A-2 and distal portions 140A-4. In one embodiment, the narrow portion 140A-6 has a diameter or width that is no more than about 60% of the diameter or width of the smallest of the proximal portion 140A-2 and distal portions 140A-4. In one embodiment, the narrow portion 140A-6 has a width that is configured to impede the distal portion 140A-4 from backing out of the LAA. In certain embodiments the proximal portion 140A-2 is optional or can have the same width as the narrow portion 140A-8. The balloon of FIG. 5A has advantages in that the non-constant diameter configuration of balloon 140A along its length prevents or reduces movement of the balloon 140A in at least one direction along the axis of the LAA. The combination of the narrow portion 140A-6 and the distal portion 140A-4 limits movement of the balloon 140A proximally out of the LAA into the LA and downstream in the heart or vasculature. The combination of the narrow portion 140A-6 and the proximal portion 140A-2 prevents or reduces movement of the balloon 140A distally into the LAA, which movement could result in some of the curable agent being exposed or released or the filling of the LAA being less complete than desired. Further, the narrow portion 140A-6 has a concave surface 140A-8 when viewed from the side. The surface 140A-8 provides a longer flow path for any curable agent to escape the LAA space prior to curing. In other words, the curable agent must flow around the distal portion 140A, along the concave surface 140A-8 and around the proximal portion 140A-2 to escape from the LAA prior to curing. Such a flow path is less likely to be traversed by a selected curable agent. The configuration of the balloon 140A can enable a wider array of curable agents to be used, for example, more viscous agents could be used in some embodiments due to the circuitous path that such an agent would have to follow to escape the LAA.

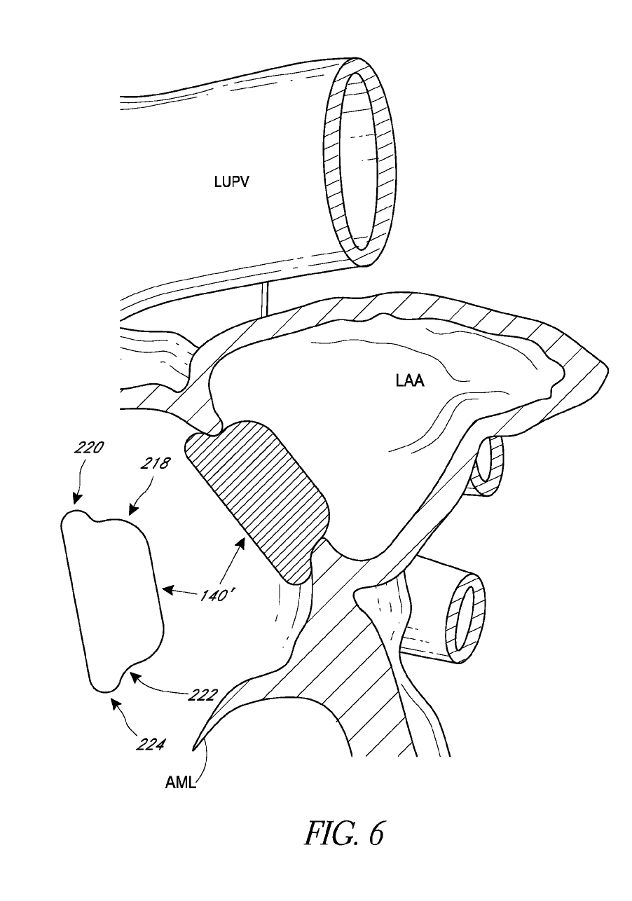

FIG. 6 shows another embodiment, in which the balloon has a distal portion 218 adapted to be positioned inside the LAA and a proximal portion 220 adapted to conform to the ostium. In FIG. 6, the balloon 140′ is shown in place (lower image) and in isolation (upper image). The proximal portion 220 can be configured to completely cover the ostium and further cover a portion of the LA around the ostium. In one embodiment, the distal portion 218 includes a generally cylindrical projection and the proximal portion 220 includes an enlarged portion configured to follow the contour of the ostium. In some cases, the proximal portion 220 is stiffer than the heart tissue surrounding the ostium and deforms this heart wall tissue into engagement with the proximal portion. For instance, a concave surface 222 can extend around the perimeter of the balloon 140′. The concave surface 222 is larger when expanded, e.g., about 10% larger, than the LAA ostium. Thus the concave surface 222 provides for sealing the contents of the LAA. A short proximal zone 224 of the balloon can extend toward the anterior mitral valve leaflet and toward the left upper pulmonary vein to enhance this sealing effect. Methods of using the system including the balloon 140′ are discussed further below.

Although it is preferred to have a very thin balloon structure, as discussed above, another approach provides a flat or high radius of curvature surface at least on the first side 212. This structure minimizes any recess for pooling of blood on the LA side of the balloon. For these embodiments, the distal side of the balloon can project farther into the LAA. This approach can reduce the volume of the LAA that must be filled with the curable agent 112 which can be an advantage.

FIG. 4 shows the use of an embodiment of the catheter device 108 placed in the representative anatomy. In particular, the elongate body 132 is shown extending up to the ostium of the LAA. The balloon 140 is expanded in the ostium providing containment or enclosure of the LAA. Containment can be achieved by providing apposition around the perimeter of the balloon 140. In some embodiments, leakage from the LAA around the balloon 140 can be reduced or minimized by applying distal pressure on the balloon 140 during a portion of procedure in which is it desired to prevent fluid from leaking. Distal pressure can be applied to the proximal portion 152, e.g., from outside the patient. As discussed below, one way of confirming minimal to no leakage is to inject contrast through the elongate body 132 into the LAA and to observe the location and/or flow of the contrast via fluoroscopy. Leakage from the LAA around the balloon 140, 140′ also can be reduced or minimized by configuring the balloon to have a zone of contact with the wall of the LA peripheral to the ostium of the LAA that is at least a minimum width.

Although the balloon 140 is a convenient way to enclose the LAA during a procedure, as discussed below, other expandable members could be used. For example, the balloon 140 can be replaced with a blocking device that is actuated by a mechanical means, such as a pull-wire. In other embodiments, sleeve can be radially enlarged by sliding an outer sheath distally over the elongate body 132, where a gasket like device is coupled at one end with the distal end of the sheath and at another end with a zone of the elongate body 132 proximal of the holes 176. Such devices have the advantage of eliminating inflation ports and lumens.

One technique for keeping the catheter device 108 from contacting the wall surrounding the LAA in a way that would potentially pierce that wall is to use an atraumatic tip, such as the pigtail member 136. Although a pigtail is illustrated in the figures, other shapes that are atraumatic and facilitate delivery of one or both of a contrast media and the curable agent 112 can be used.

As noted below, the elimination or reduction in volume of the LAA can be achieved by delivering a polymer into the LAA and curing it in situ. To maintain the security of the engagement of the curable agent 112 when partially or fully cured, it is preferred to reduce or minimize disruption of the pigtail member 136. One strategy for reducing or minimizing disruption is not to attempt to remove the pigtail portion after the polymer or other sealant is delivered.

In some embodiments, a disengageable structure such as the connection hub 144 can be provided. The connection hub 144 can be disposed along the elongate body 136 as shown in FIGS. 2-3A. In general, the connection hub 144 has a first configuration in which the proximal portion 152 of the elongate body 136 is coupled with the pigtail member 136. The connection hub 144 has a second configuration in which at least the pigtail portion 136 is un-coupled from the proximal portion 152 of the elongate body 136. The in-situ curable polymer or other flowable agent or sealant is adapted to be delivered through the lumen 172 of the elongate body 136, out of the outlet port(s) 176 between the distal end 180 and the balloon 140.

FIGS. 2A and 3A illustrate embodiments of the connection hub 144 that retain a low profile while enabling disconnection of the proximal portion 152 from the pigtail 136. The connection hub 144 can be operated by actuating a structure at the proximal end of the elongate body 132. The hub 144 can include a proximal portion 144A and a distal portion 144B. Preferably the proximal and distal portion 144A, 144B can be connected and disconnected in a convenient manner. Such connection and disconnection can be facilitated with a mechanism or can be triggered by an event during the process.

In the illustrated embodiment a plurality of threads 250A are disposed on the outside of the distal end of the proximal portion 152. A corresponding plurality of threads 250B are disposed within a recess 254 formed at the proximal end of the distal portion 156. As discussed further below, the threads are initially engaged prior to procedure. At a stage of the procedure after the curable agent 112 is delivered into the LAA, the threads 250A, 250B are disengaged from each other such that the distal portion 156 can be separated from the proximal portion 152. Upon separation, the proximal portion 152 can be withdrawn from the distal portion, leaving the distal portion in place. In one technique, the curable agent 112 is at least partially solidified and holds the distal portion 156 in place. The proximal portion 152 can then be torqued from outside the patient to cause the threads 250A, 250B to disengage.

In one variation, the threads 250A are disposed in a recess of the proximal portion 152 and threads 250B on an outer surface of the distal portion 156. In another variation, the threads 250A, 250B are replaced by a structure that enables disengagement by minimal to no torque applied by the user. For instance, FIG. 2B illustrates a connection hub that can be provided to allow disconnection of the distal portion 156 based on a simple operation at the proximal end of the catheter device 108. The connection hub includes a control track 250C and a control pin 250D. The control pin 250D can be a short lateral protrusion extending away from the outside surface of the distal portion 156. The control track and pin 250C, 250D are configured to allow specific movements of these structures relative to each other. The track 250C can have a J shape so that the pin 250D rests at a closed end of the track when the proximal and distal portions 152, 156 are engaged. To disengage these structures, the pin 250D can be caused to travel through the track 250C initially proximally along a first short leg of the track, then circumferentially along a second leg of the track transverse to the first leg, then distally along a third leg of the track transverse to the second leg. FIG. 2B show the pin rotated 180 degrees so that both the track and the pin are visible. It will be understood that the track and pin 250C, 250D will be on the same side of the catheter device 108 when engaged. The length of the second portion of the track 250C determines the amount of rotation of the proximal and distal portions 152, 156 needed to disengage the distal portion from the proximal portion. The rotation can be a small amount, e.g., less than about 45 degree, in some cases less than 20 degrees, and in further embodiments less than about 10 degrees.

Preferably the movements of the proximal portion 152 relative to the distal portion 156 are controlled by simple movements at the proximal end of the catheter device 108. For instance a push button can cause the proximal portion 152 to travel axially a short distance, e.g., corresponding to the length of the first leg or portion of the track 250C. A rotation device can cause the proximal portion 152 to rotate relative to the distal portion 156, e.g., a distance corresponding to the length of the second leg or portion of the track 250C. The rotation device can include a ramped surface that pushes the catheter body in the proximal portion 152 to urge the catheter body to rotate about the longitudinal axis of the catheter body. Finally a translation device can be provided to move the control pin 250D axially along the third leg or portion of the control track 250C. In this mechanism, the movements described can be relative motions, in which one of the proximal and distal portions 152, 156 is stationary or in which both proximal and distal portions are moving, either simultaneously or sequentially.

Other remotely disengageable connections or locking devices can be placed at the connection hub 144.

FIGS. 2 and 3 show that the connection hub 144 can be located distal of or proximal of the balloon 140 in various embodiments. When located distal of the balloon 140, the contents delivered into the LAA will be exposed at least initially to the LA after curing or solidifying. When located proximal of the balloon 140′, the contents delivered into the LAA will be covered by or capped by the balloon.

### II. Curable Agents

As noted above, the system 100 is configured to occlude the LAA by causing a space filling mass to be formed in the LAA. In some cases, the space filling mass is therafter exposed in the LA at least initially. In other embodiments, a structure is filled with a medium that solidifies, where the structure forms a cap to separate the curable agent from the LA.

A. Example of Curable Agents for Filling the LAA

The suitable space filing mass may include a polymeric biomaterial that is produced by polymerizing or cross-linking two or more components of a curable agent in vivo. In accordance with certain embodiments, preferred curable agent has the following characteristics: (1) low viscosity for catheter delivery; (2) short curing time at body temperature, without significantly raising the temperature during curing; and (3) radio-opacity to allow fluoroscopic imaging during delivery. The curable agents that will be used to fill the LAA should also have minimal toxicity and biocompatibility, as the agent may be in contact with the blood stream or tissue of a patient. Once the curable agent is cured and in place, the cured biomaterial should also have a long term chemical stability in aqueous and biological environments. In some embodiments, the cured material exhibits long-term stability (preferably on the order of at least 3 years, at least 5 years, at least 8 years or at least 10 years in vivo).

Some embodiments provide injectable hydrogels as curable agents. The hydrogels may be natural or synthetic hydrogels. Natural hydrogels may include, but not limited to, fibrin sealant/glue, alginate, composite hydrogels comprising fibrin and alginate, etc. Synthetic hydrogels may include, but are not limited to, dextran grafted poly(caprolactone)-2-hydroxyethyl methacrylate (PCL-HEMA) and copolymerized with poly(N-isopropylacrylamide) (PNI-PAAm), α-cyclodextrin and ploy(ethylene glycol) (MPEG-PCL-MPEG) triblock copolymer, vinyl sulfone derivatized PEG (PEG-VS) combining with dithiothreitol (DTT), etc. These hydrogels are described in more details in Tous, E., et al., “Injectable Acellular Hydrogels for Cardiac Repair,” J. of Cardiovasc. Trans. Res. (2011) 4:528-542, the content of which is incorporated by reference herein.

One example of the injectable hydrogel includes a fibrin sealant. The fibrin sealant has been used as an adjunct to hemostasis, wound healing, tissue adhesion, and etc. One example is Tisseel VH fibrin sealant (Baxter Healthcare Corp, Deerfield, Ill.). The formation of fibrin involves a two-step process. Fibrinogen is a glycoprotein, and through the action of activated thrombin, the fibrinogen molecule is cleaved of peptides and converted into a soluble monomer. The fibrinogen monomers are cross-linked into an insoluble fibrin matrix by the action of activated factor XIII. The formulations of fibrin sealant include fibrinogen and an antifibrinolytic agent, such as aprotinin. The details of the fibrin sealant can be found in MacGillivray, T. E., “Fibrin Sealants and Glues,” J Card. Surg. 2003; 18:480-485 (“MacGillivray”), the content of which is incorporated by reference herein. In some embodiments, the fibrin sealant may also be home-made e.g., manufactured or prepared in the field (also described in MacGillivray).

In some embodiments, in-situ forming hydrogels may be designed and formed by chemical crosslinking, such as Michael-type addition, radical polymerization, or enzymatic crosslinking. Michael-type addition reaction involves mixing aqueous solutions of polymers bearing nucleophilic (amine or thiol) and electrophilic groups (vinyl, acrylate, or maleimide) to obtain an in-situ forming hydrogel. Radical polymerization can be used to prepare robust and stable hydrogels by creating radicals from initiator molecules through thermal, redox or photo-initiated mechanisms, then the radicals propagate through unreacted double bonds during polymerization to form long chains, and the chains react with each other to form crosslinked polymeric networks. Enzymatic crosslinking includes employing enzymes like horseradish peroxidase (HRP) or tyrosinase to form hydrogels. More details on the in-situ forming hydrogels can be found in Jin, R., “In-Situ Forming Biomimetic Hydrogels for Tissue Regeneration,” Biomedicine, pages 35-39, the content of which is herein incorporated by reference.

Other embodiments provide injectable composition adapted to form a space filling mass in situ. For instance, a combination of solidifying and contrast agents could be provided. The solidifying agent can include an ethylene vinyl alcohol copolymer or the like and a biocompatible solvent, such as dimethylfuloxide (DMSO). Concentrations of ethylene vinyl alcohol copolymer can vary from about 6% to about 8%. The amount of ethylene vinyl alcohol copolymer may affect viscosity. Additional embodiments of this nature are discussed in U.S. Pat. No. 5,667,767, the content of which is herein incorporated by reference.

Other embodiments provide injectable medical grade adhesives. For instance, an appropriate biocompatible formulation of cyanoacrylate or other acrylics could be used. In other embodiments a two part composition, such as an epoxy could be used. Other embodiments may include adhesive made of other suitable materials, such as silicone and polyurethane.

Any of adhesives, solidifying agents, or curable agent or the like can include one or more contrast agents to enhance visibility using conventional imaging techniques. Suitable contrast agents preferably are water insoluble. The contrast agent can include any one of or a combination of tantalum, tantalum oxide, or barium sulfate. In one formulation, the contrast agent includes micronized tantalum powder. Other contrast agents can be used that may be to some extent soluble so long as they remain at the LAA site during initial placement.

B. Example of Curable Agents for Forming a Cap Member

In addition to the curable agents described above, other curable agents suitable for use as an inflation medium can also be employed to form a cap member such as any of the balloons discussed above. The cap member may be inflated using a variety of inflation media. Useful inflation media generally include those formed by mixing multiple components curable agents. Although it is preferable that the inflation media is biocompatible, the degree of biocompatibility does not need to be to the same extent as the curable agents for filing the LAA.

Details of compositions suitable for use as an inflation medium in a cap member are described in greater detail in U.S. patent application Ser. No. 09/496,231 to Hubbell et al., filed Feb. 1, 2000, issued as U.S. Pat. No. 7,744,912, entitled “Biomaterials Formed by Nucleophilic Addition Reaction to Conjugated Unsaturated Groups” and U.S. Pat. No. 6,958,212 to Hubbell et al. The entireties of each of these patent documents are hereby incorporated herein by reference.

### III. Methods and Procedures

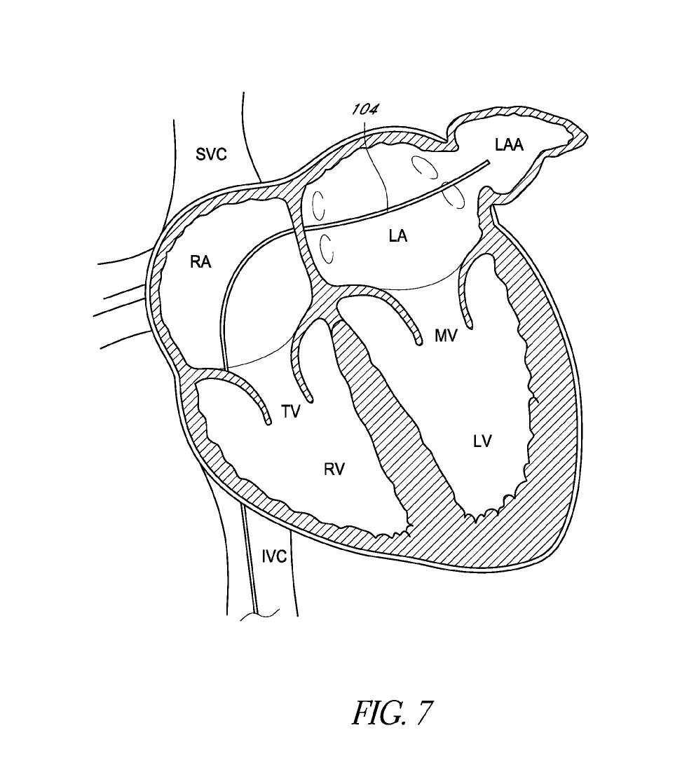

FIGS. 7-13 illustrate various steps of methods that can be used to reduce or eliminate the LAA in a patient. These methods can result in partial or full occlusion of the LAA. As discussed above, these methods provide the stoke reduction benefits of occlusion of the LAA while reducing, minimizing or eliminating the risk of puncture of the wall of the LAA.

FIG. 7 illustrates an initial portion of a procedure in which access is provided to the LAA. Peripheral venous access can be provided in a conventional manner. Thereafter an access catheter or device can be advanced through the venous vasculature to the heart. The access device can be advanced into the RA. In one technique, the access device is directed to cross the atrial septum such that the distal end thereof is in the LA. Access from the RA to the LA can be via any conventional technique, such as by puncturing the atrial septum between the LA and RA.

The access device and/or the guidewire 104 can be advanced into the LAA and held in place at that location in initial portions of the procedure. Thereafter the catheter device 108 can be advanced along the guidewire 104 until the pigtail member 136 or other atraumatic tip structure is disposed in the LAA.

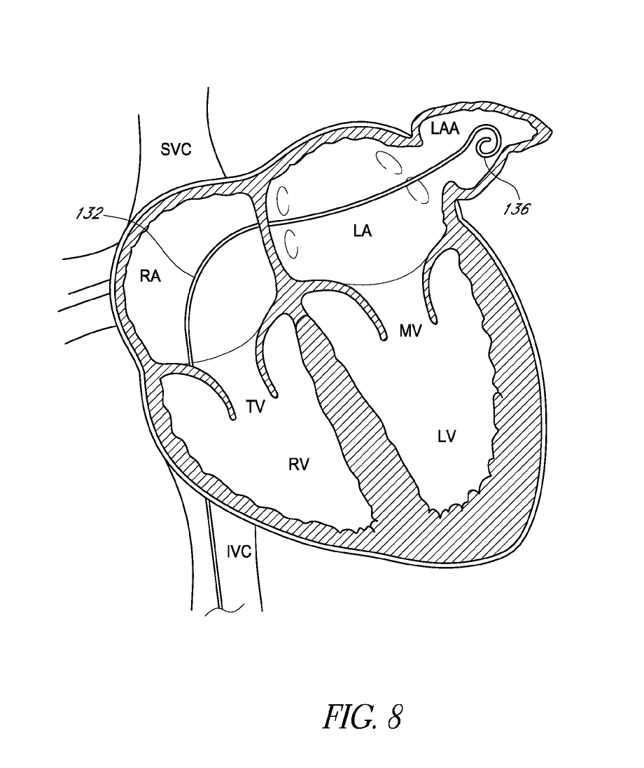

FIG. 8 shows the pigtail member 136 in a relaxed state. The pigtail member 136 is an example of a device that provides soft, atraumatic interaction with the LAA. Such a device can have a curved section that projects distally from a straight portion of the elongate body 132. The curved portion can be sized to be received fully into the LAA, e.g., the proximal end of the curve distal the ostium of the LAA. Preferably the pigtail member 136 or other atraumatic device has a width that enables two point contact interior walls of the LAA. This structure allows the catheter device 108 to be brought to rest against the LAA. This allows the clinician to apply gentle pressure to the proximal end with assurance that the ports 176 are disposed in the LAA. In preferred methods, the port 176 or the proximal most port 176 if more than one are present is or are all at or distal the ostium of the LAA.

As discussed above in connection with FIG. 2, the pigtail member 136 or other atraumatic structure can be configured to be straightened by the guidewire 104 and to transform to the curled shape when deployed. In one method, the pigtail member 136 is positioned in the LA. The distal end 180 is advanced distal the ostium to the LAA. Thereafter the wire 104 is withdrawn permitting the member 136 to curl back upon itself. The curling of the tip 136 positions the curved portion that is distal-most in the deployed configuration (e.g., as in FIG. 8) at the ostium. The pigtail member 136 can then be advanced into the LAA.

In one technique, a portion of the procedure involves evaluating the configuration of the LAA. One approach is to inject a contrast agent through the lumen 172, out of the ports 176 into the LAA. This step can involve injecting a standard contrast medium, which need not be trapped or captures. This portion of some procedures can be performed prior to enclosing the LAA. In other techniques it may be desirable to capture the contrast medium. In such case, the elongate body 132 can be provided with one or more aspiration lumens. In another embodiment positive and negative pressure can be alternately applied to the lumen 172.

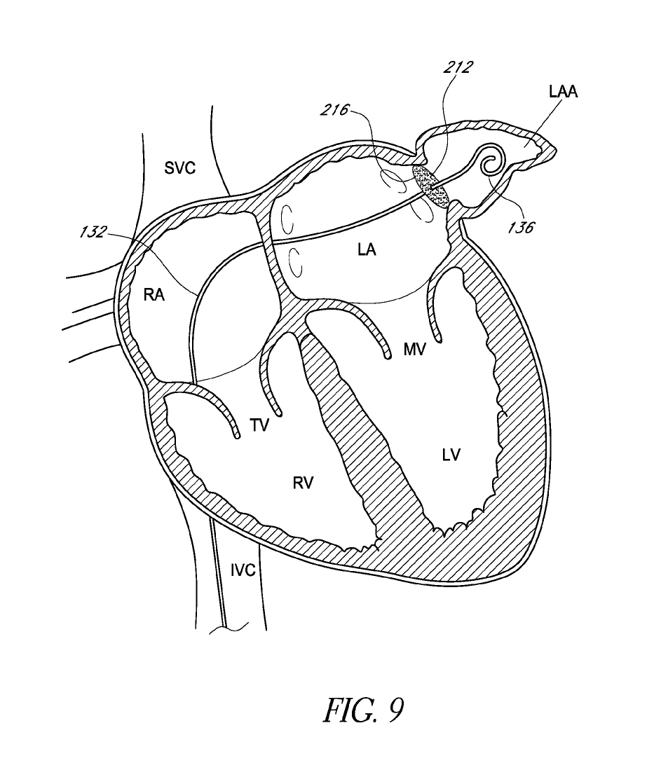

In some procedures, after the LAA has been evaluated an expandable member, such as the balloon 140 can be deployed as shown in FIG. 9. The balloon 140 can be inflated using an inflation medium delivered through the lumen 200. In embodiments illustrated by FIGS. 3-6 it may be convenient to inflate the balloon 140′, 140A with a material that will also solidify to retain the balloon in the enlarged state permanently. The balloon 140′ is preferably deployed distal the ostium of the LAA. The balloon 140 can be inflated to provide an outer perimeter that overlaps with the ostium of the LAA by an amount sufficient to provide a good seal in combination with a radially outward or a distally directed pressure. A method illustrated by FIG. 5 involves inflating the balloon within the LAA. A method illustrated by FIG. 6 involves overlapping at least a portion of the ostium toward and in some cases into the LA. Methods related to FIG. 6 advantageously eliminate the entire volume of the LAA. The balloon 140 preferably seals the ostium of the LAA or at least a majority of the volume of the LAA from the LA. For most patients, the ostium of the LAA is at least somewhat deformable. Thus, the balloon can be made stiff when inflated at least on the first side 212 such that distal pressure causes the tissue forming or disposed above the LAA ostium to conform to the balloon surface. This conformance can be in an annular area having the overlap dimension. As used herein in this context “seals the ostium of the LAA” is defined as eliminating or substantially reducing leakage of contrast when contrast is injected through the lumen 172 at typical ambient pressures generated in the LAA.

In some cases, clinically effective procedures can be performed even if the balloon 140 can provide a lesser seal. For example, where the sealant has higher viscosity, it may exert less pressure on the balloon 140 tending to open gaps at the ostium and/or is too viscous to flow through small gaps that may leak less viscous contrast media.

Some procedures may include confirming that the LAA is sealed. The balloon 140 can be inflated while a contrast medium is injected through the lumen 172. This approach is useful where distal pressure is more variable and the clinician wishes to confirm that distal pressure and/or the overlap of the balloon 140 is sufficient or correct. In some embodiments, the balloon 140 may be asymmetric, e.g., having a height greater than a width for non-circular ostium of the LAA. In such cases, if the short axis of the balloon 140 is aligned with the long axis of the LAA, there may be significant leakage. Thus, alignment of the balloon 140 can be enhanced and leakage reduced by injecting contrast to confirm that the balloon 140 is properly aligned and/or a sufficient seal is achieved.

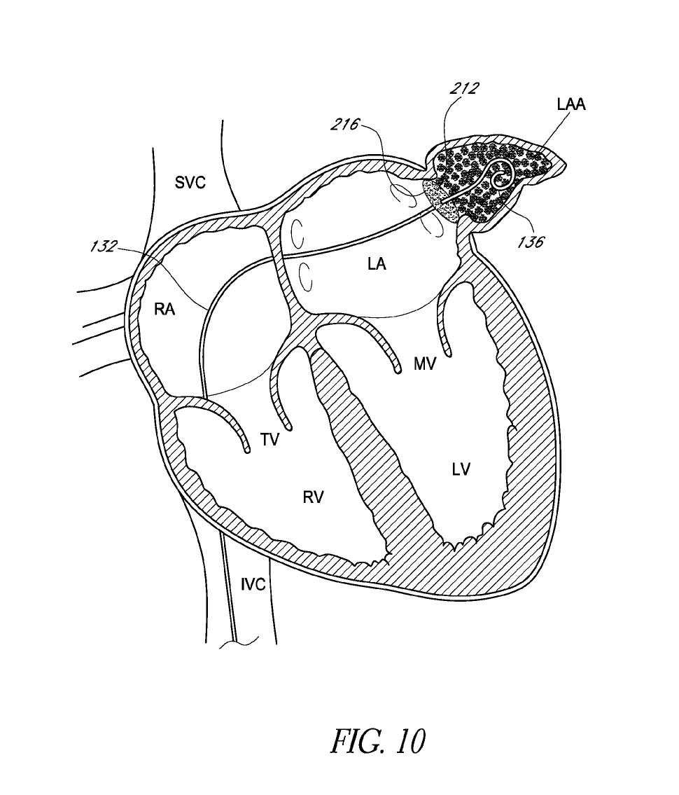

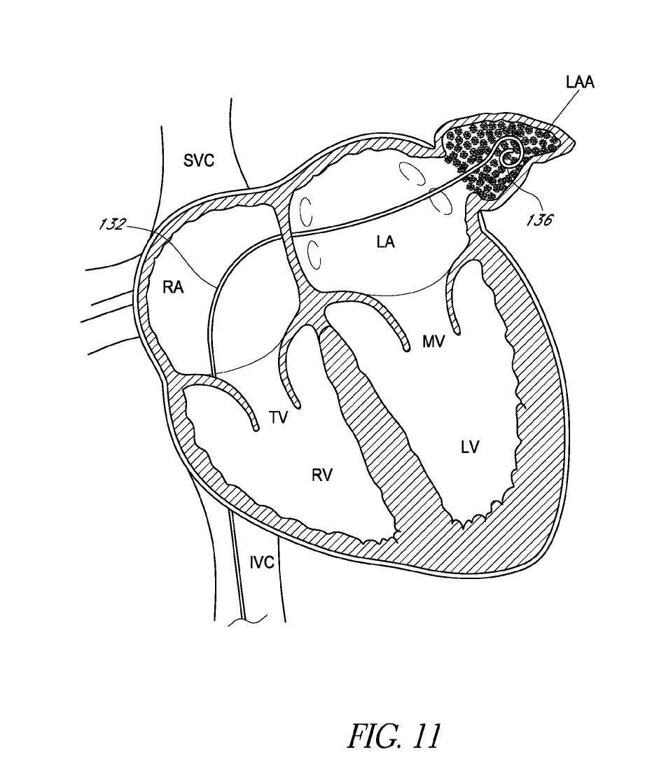

After the balloon 140 is inflated and optionally a seal is confirmed, a sealant or other agent can be caused to flow through the procedure catheter 108 as shown in FIG. 10. The sealant flows out of the plurality of outlets 176 formed in the atraumatic tip 136 into the LAA. As the sealant flows it may fill the LAA from the distal end toward the balloon 140 or may flow from the outlets 176 proximally and distally in the LAA. In either case, as the LAA fills, the clinician may notice some pressure acting against the proximal portion 152 of the elongated body 132 that corresponds to pressure of the curable agent 112 on the first side 212 of the balloon 140. In some techniques, the curable agent 112 include, or is combined with a contrast medium. In one combination the curable agent 112 and contrast are mix together so that any visible contrast on fluoroscopy indicates time to cease injecting sealant. In other approaches, the curable agent 112 and a contrast medium may be injected alternately through the lumen 172 into the patient so that the clinician can monitor the filling of the LAA. In particular, the degree of fill can be visualized in this approach if the curable agent 112 is not itself radiopaque.

Once the LAA has been sufficiently filled, the curable agent 112 can be cured. This can be achieved in any suitable manner. In one approach, the curable agent 112 includes a sealant is self-curing over a short time. In another embodiment, a catalyst can be injected through the elongate body 132, e.g., through the lumen 172 to mix with the sealant to cause it to cure. Any other form of catalyst can be used, including delivering heat through the catheter or absorbing heat from the patient. FIG. 11 shows a cured state. In the embodiment of FIG. 2, the balloon 140 is deflated but would still be present. The balloon 140 can be deflated without concern for embolization after the agent 112 is cured because the agent is not able to flow or otherwise be liberated from the LAA. In FIG. 11 the balloon is not shown for clarity.

In some techniques, it may be preferred not to disrupt the curable agent 112 as it cures or after it cures. Accordingly, in one technique the pigtail member 136 is detached from the proximal portion 152 of the elongate body 132. The proximal portion 152 of the elongate body 132 can be torqued to disengage the threads 250A from the threads 250B. Because the pigtail member 136 is lodged in the curable agent 112, once the curable agent is cured, the distal portion 144B of the connection hub 144 is held stationary while a torque applied at the proximal end causes the proximal portion 144A of the hub to rotate.

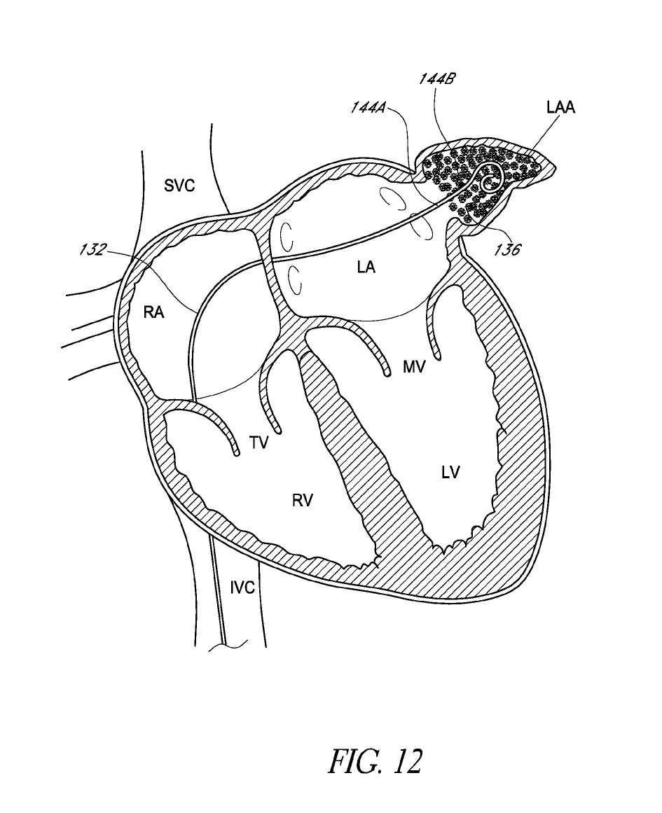

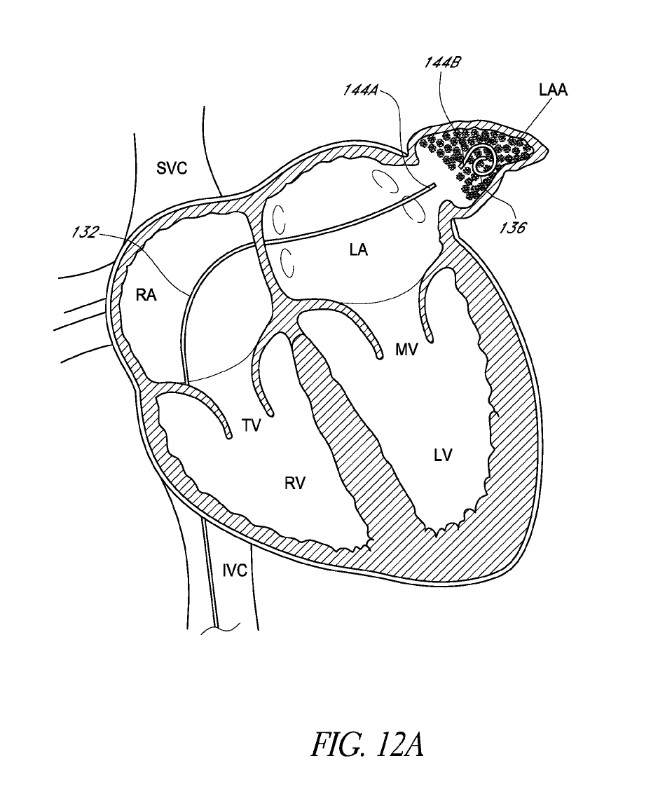

FIG. 12 shows one approach where the curable agent has been held in place up to the location of the ostium and permitted to cure. This approach provides a generally flush configuration in which little to no recessed space is provided in the vicinity of the LAA after treatment. This approach is more prone to embolization of the curable agent because the curable agent is disposed adjacent to normal blood flow through the LA. Accordingly, it may be preferred to provide additional curing time prior to removing the expandable member (which may be functioning like a cap) and also prior to detaching the pigtail member 136. In another approach the expandable member is expanded more distally within the LAA. FIG. 12A shows the configuration of the cured curable agent in this placement. The exposed face of the cured curable agent is located somewhat distal of the ostium. This approach reduces the risk of embolization of the curable agent for several reasons. The expandable member can be lodged in the ostium during the flowing in of the curable agent. In some patients, the ostium provides a narrows between deeper reaches of the LAA and the atrium. This narrows can constrain the expandable member when the expandable member is expanded distally of the narrows but in contact with the narrows. This constraint minimizes or prevents proximal shifting of the expandable member. Accordingly, the expandable member will not be likely to slip in the LAA. The stable position will minimize leakage of the curable agent before it cures. Also, the exposed face of the cured curable agent is more remote from the normal flow of blood in the atrium and thus less likely to be eroded or otherwise affected by the flow. While some space in the LAA remains un-occluded the volume of the LAA is substantially decreased to provide clinical benefit.

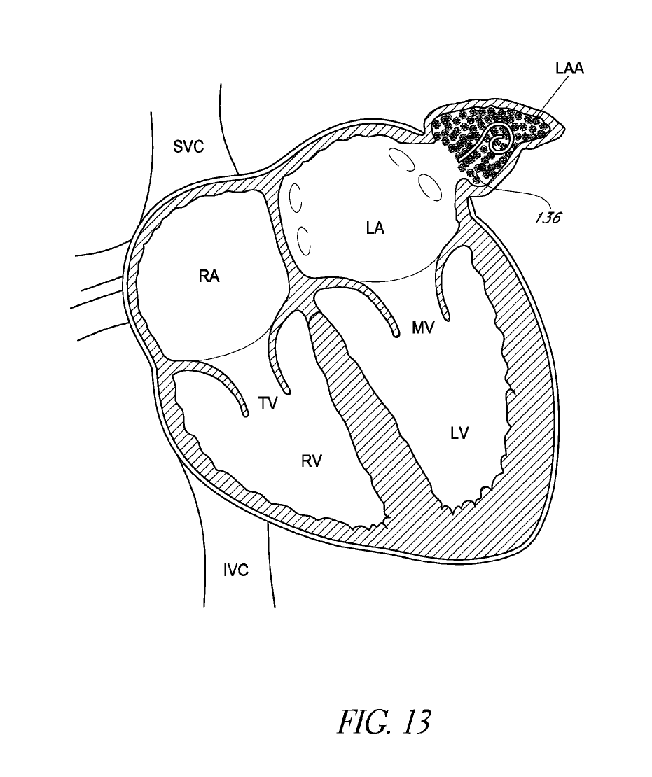

As noted above, FIG. 13 shows a stage subsequent to that of FIG. 12 in which the proximal portion of the catheter device of FIGS. 2-2A has been removed from the patient. As shown, this stage leaves the atraumatic tip including the pigtail member 136 in place in the heart.

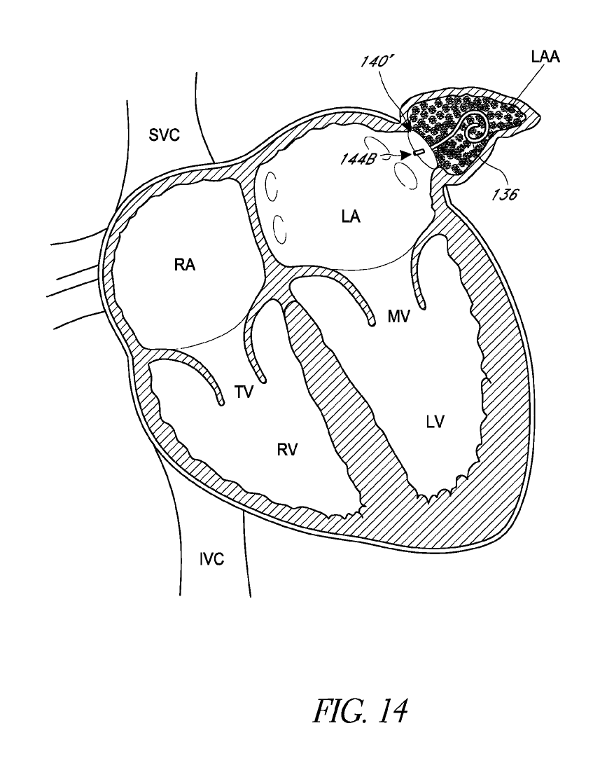

FIG. 14 illustrates aspects of methods related to the embodiments illustrated in FIGS. 3 and 3A. In these methods, the balloon 140′ and the pigtail member 136 are both detached so that the curable agent 112 is not disrupted and is covered so that blood does not come into contact with the agent. FIG. 14 shows that a portion of the connection hub 144 can project away from the surface 216 of the balloon 140′ toward the LA in some embodiments. In some cases, the balloon 140′ is positioned far enough into the LAA that the distal portion 144A of the connection hub 140 does not extend into the LA to be within the flow of blood in the LA. In some embodiments, the distal portion 144A of the connection hub 144 is recessed at least partially into a portion of the balloon 140′ at least when the balloon 140′ is inflated and the proximal portion 144B is detached from the distal portion 144A. Thus, the balloon 140′ can be placed at or close to the ostium of the LAA without causing the distal portion 144A to project into or the LA by a significant distance. It may not be necessary for the curable agent 112 to remain in a sealed space after it cures, so the balloon 140′ could be permitted to over a short or longer period release the inflation medium at which time it may just lie flat against the cured agent. As noted above, Dacron or other structure configured to encourage endothelialization or similar biological processes encasing foreign matter can be provided. Thus, the balloon 140′ whether remaining inflated or not may eventually be covered by a natural barrier.

As discussed in connection with FIG. 12A, the balloon 140′ could be positioned distal of the ostium in one technique. This distal placement would cause the balloon 140′ to be lodged distally of any narrows in the vicinity of the ostium. The lodging of the balloon in this space reduces or eliminates the chance that the balloon will be embolized after detachment from the catheter body. Another technique for reducing or eliminating the chance of embolization of the balloon is to permit the curable agent to cure sufficiently such that the balloon is sure prior to detachment from the proximal portion of the catheter.

Although the present invention has been disclosed with reference to certain specific embodiments of devices and methods, the inventors contemplate that the invention more broadly relates to methods disclosed above, such as those useful for orienting a catheter with respect to an anatomical structure, as well as performing diagnostic and/or therapeutic procedures in the heart or adjacent the heart. Accordingly, the present invention is not intended to be limited to the specific structures and steps disclosed herein, but rather by the full scope of the attached claims.

1. A catheter device, comprising:
an elongate body with a fluid flow lumen extending therethrough, the fluid flow lumen being in fluid communication with an outlet port adjacent to a distal end of the elongate body;
an atraumatic member disposed at the distal end of the elongate body;
an expandable member disposed proximal of the atraumatic member, the expandable member configured to block an opening of the left atrial appendage (LAA);
a locking device disposed adjacent to the expandable member having a first configuration in which the elongate body is coupled with the atraumatic member and second configuration in which the elongate body is un-coupled from the atraumatic member.

2. The catheter device of claim 1, wherein the expandable member comprises a balloon.

3. The catheter device of claim 2, wherein the balloon has an unconstrained diameter of about 10% greater than the width of the LAA.

4. The catheter device of claim 2, wherein the balloon has a distal portion and a narrower portion proximal of the distal portion, the narrower portion configured to receive an anatomical narrows at the ostium between the left atrium and the LAA.

5. The catheter device of claim 4, wherein the balloon has a distal portion with a first diameter, a proximal portion with a second diameter and a narrow portion disposed between the proximal and distal portions.

6. (canceled)

7. The catheter device of claim 1, wherein the locking device comprises a threaded interface between the expandable member and the atraumatic member.

8. (canceled)

9. The catheter device of claim 1, wherein the lumen is in flow communication with a liquid sealant port and a contrast media port at the proximal end of the elongate body.

10. The catheter device of claim 1, wherein the locking device is disposed between the expandable member and the atraumatic member.

11. The catheter device of claim 1, wherein the locking device is disposed proximal of the expandable member.

12. (canceled)

13. (canceled)

14. (canceled)

15. A method for occluding a left atrial appendage (LAA) of a heart, the method comprising:
advancing an access catheter through the venous vasculature into the right atrium;
puncturing the septum between the left and right atria;
advancing a guidewire through the septum into the LAA;
advancing an atraumatic member of a procedure catheter along the guidewire and into the LAA such that a plurality of outlets formed in the atraumatic member are distal the ostium of the LAA;
evaluating the configuration of the LAA;
deploying an expandable member from the procedure catheter to seal the LAA ostium;
confirming the sealed state of the LAA ostium;
flowing a sealant through the procedure catheter and out of the plurality of outlets formed in the atraumatic member into the LAA;
after the sealant is secured in the LAA, detaching the atraumatic member from a proximal portion of the procedure catheter.

16. The method of claim 15, wherein the atraumatic member comprises a pigtail tip having a length of between about 15 mm to about 40 mm.

17. The method of claim 15, wherein the atraumatic member comprises a pigtail tip having a diameter of between about 4 and about 16 French.

18. The method of claim 17, further comprising injecting contrast through the pigtail in conjunction with evaluating the configuration of the LAA.

19. The method of claim 15, further comprising performing a trans esophageal or intracardiac echocardiographic analysis to assess the configuration of the LAA and to confirm placement of at least one of the atraumatic member and the expandable member.

20. The method of claim 15, wherein detaching the atraumatic member comprises disengaging a mechanical connection between the atraumatic member and a proximal portion of the procedure catheter.

21. The method of claim 20, further comprising unscrewing the atraumatic member from the proximal portion of the procedure catheter.

22. A method for occluding a left atrial appendage (LAA) of a heart, the method comprising:
positioning a guide member through the heart and into the LAA;
advancing an occlusion catheter system having an atraumatic member disposed at a distal portion thereof along the guide member such that the distal portion including the atraumatic member is disposed within the LAA;
injecting a fluid sealant through the occlusion catheter system into the LAA;
permitting the fluid sealant to solidify in the LAA around the atraumatic member to minimize flow of blood into the LAA; and
detaching a proximal portion of the occlusion catheter system from the atraumatic member.

23. The method of claim 22, further comprising deploying an expandable member laterally from the occlusion catheter system into opposition with the ostium of the LAA prior to injecting the fluid sealant.

24. The method of claim 23, further comprising confirming adequate apposition of the expandable member with the ostium prior to injecting the fluid sealant.

25. The method of claim 24, further comprising injecting contrast media into the LAA and observing any leakage prior from the LAA prior to injecting the fluid sealant.
